(19) United States
(12) Patent Application Publication
(10) Pub. No.: US 2015/0148852 A1
(43) Pub. Date: May 28, 2015

(54) BIONIC ELASTIC FIXING DEVICE

(71) Applicant: THE THIRD HOSPITAL OF HEBEI MEDICAL UNIVERSITY, Shijiazhuang, Hebei (CN)

(72) Inventors: Yingze Zhang, Shijiazhuang (CN); Lin Wang, Shijiazhuang (CN); Wei Chen, Shijiazhuang (CN); Yueju Liu, Shijiazhuang (CN)

(21) Appl. No.: 14/406,375

(22) PCT Filed: Jun. 6, 2013
(86) PCT No.: PCT/CN2013/076840

§ 371 Date: Dec. 8, 2014

(30) Foreign Application Priority Data
Jun. 8, 2012 (CN) 201210188096.X
Jun. 8, 2012 (CN) 201220269629.2

(51) Int. Cl. A61B 17/84; A61B 17/86

(52) U.S. Cl. CPC A61B 17/842; A61B 17/86; A61B 2017/00862
USPC 606/328

(57) ABSTRACT

A bionic elastic fixing device relates to orthopedic medical devices, and is used to implement bionic elastic internal fixing for joint dislocation. The device consists of an elastic component (1) and a fixing assembly (2, 3, 4). The elastic component (1) may be multiple strip-shaped elastic mesh pieces, and has the assembly direction being the same as the ligament fibers at the joint required to be fixed. The elastic modulus of the elastic component (1) matches the elastic modulus of the ligament fibers at the joint required to be fixed. Two ends of the elastic component (1) are fixedly connected to the dislocated joint by the fixing assembly (2, 3, 4). The elastic component (1) has the same shape as the ligament fibers at the joint required to be fixed.

### FIELD OF THE INVENTION

The present invention relates to a device to implement internal fixing for joint dislocation in the treatment of orthopedic injuries, belonging to the technical field of orthopedic medical device.

### BACKGROUND OF THE INVENTION

Joint dislocation is a common injury in the orthopedic therapy. The fixing on the dislocated joints is the most important part for the treatment. The present fixing for joint dislocation used in clinical treatment is firmly fixing, which does not conform to the law of biomechanics, with poor postoperative result, thus, it is necessary to be improved. In the following, it will be described in details using examples of fixing of joint dislocation and separation.

Acromioclavicular joint is an amphiarthrosis; when lifting and moving 0°-30° or exceeding 135° of abduction of shoulder joint, acromioclavicular joint can move slightly at 20° to complete the fine movements of the upper extremities. Clinically, Acromioclavicular joint dislocation is one of the common injuries, and its surgical techniques include over thirty methods such as clavicular hook plate, suture anchors, Kirschner wire tension bands and Endobutton internal fixation, etc. Among these methods, the treatment of joint dislocation by clavicular hook plate can achieve excellent effect, but the surgery may produce trauma and severe soft tissue injury. The clavicular hook plate passes through acromion, which is prone to produce subacromial impingement when extending upper limbs, moreover, it is expensive. Kirschner wire tension bands has the defects such as coming off, insecure fixing, which may cause instability, etc. In particular, for these internal fixing methods, the acromioclavicular joint that slightly move is strongly fixed, which breaks the biomechanical laws; and after removal of the internal fixation, the acromioclavicular joint dislocation has a higher recurrence rate and patient's shoulder functions are often restricted.

Inferior tibiofibular joint consists of syndesmosis, plica and bone joint. The inferior tibiofibular joint structure is both firm and elastic, which is important to ensure the normal movement of ankle joint. The inferior tibiofibular syndesmosis plays an important role in maintaining stability of ankle joint. The inferior tibiofibular fibrous joint composed of anterior and posterior ligaments of inferior tibiofibula is essentially an amphiarthrosis, which plays important role in regulating the fine movement of ankle joint. Ankle fracture and dislocation is one of the most common clinical fractures and dislocations, and nearly 60% patients with ankle fracture and dislocation are complicated with tibiofibular separation. Tibiofibular separation is a common clinical injury, which may lead to ankle instability. Due to the changes to the contact area of ankle and its biomechanical characteristics, it possibly leads to secondary osteoarthritis, and aggravate the injury to ankles. The treatment on inferior tibiofibular syndesmosis injury should maintain the elasticity fixation. At present, the early surgical fixation of acute inferior tibiofibular separation is still the main treatment means. The commonly used surgical technique is to fix with lag screws after reduction of inferior tibiofibular joint; but this fixation is strong, which does not accord with the biological mechanical characteristics of inferior tibiofibular joint; thus, elastic fixation should be performed.

Symphysis pubis is composed of symphysial surface on both sides connected by fibrocartilages. The superior and inferior surfaces and anterior face are reinforced by ligaments. The superior part is superior pubic ligament and the inferior part is arcuate pubic ligament. The symphysis pubis separation is a common pelvic injury. The symphysis pubis is also an amphiarthrosis same as the above joint structure. The characteristics of slight movement of joint are particularly important in the natural childbirth in women. Currently, the treatment on injuries including pubic symphysis separation mainly includes internal fixation treatment for purpose of anatomic reduction and rigid fixation and external fixation treatment for the purpose of maintaining the stability through fast treatment. None of the two treatment strategies considers the structural and biomechanical characeteristics of symphysis pubis as amphiarthrosis. It is prone to generate local irritation of internal fixation and the insecure external fixation after treatment.

Distal radioulnar joint is enarthrosis composed of head of ulna and sigmoid cavity of radius, which plays an important role in maintaining the forearm rotation and forearm mechanotransduction. Interosseous membrane, dorsal and volar ulnar ligaments are important factors for maintaining the stability of distal radioulnar joint. Joint dislocation and ligament injury is the important reasons for wrist pain and functional limitation. The main treatment protocols for dislocation of distal radioulnar joint or injuries of surrounding ligaments are conservative treatment, suture of capsular ligament and alternative autologous tendon displacement, etc. The conservative treatment cannot effectively restore the continuity of ligaments; through healing of scars, the force maintaining joint stabilization reduces. The suture of capsular ligaments often shortens the ligament and even contracture, restricting the joint functions.

Based on the structure and biomechanical characteristics of the amphiarthrosis as well as the limitation of the above treatment strategies, we designed the bionic elastic fixing device for treatment of amphiarthrosis injury according to the ligament direction, structural characteristics and biomechanical characteristics, etc.

### SUMMARY OF THE INVENTION

A kind of bionic elastic fixing device is disclosed in the present invention. Such fixing device can simulate the physiological state of the dislocated joints to achieve slight movement and the optimal treatment effect, having such advantages as quick post-surgery recovery, low recurrence rate of dislocation and desirable functional recovery of the body part of the patient where dislocation occurs.

The technical scheme to solve the above technical problems is described as follows:

A bionic elastic fixing device comprises an elastic component and a fixing assembly that fixedly connects the two ends of the elastic component and the dislocated joint required to be fixed. The said elastic component is of strip-shaped, and its assembly direction is the same as the ligament fibers at the joint required to be fixed. The elastic modulus of the elastic component matches the elastic modulus of the ligament fibers at the joint required to be fixed. The fixing assembly is fixedly mounted to the two ends of the elastic component.

For the above bionic elastic fixing device, the said elastic component is multiple elastic cables, elastic mesh pieces or elastic bands.

For the above bionic elastic fixing device, the length of the said elastic component is the same as the length of ligament fiber at the joint required to be fixed, and the fixed points on two ends of elastic component are the same as the growing points of the two ends of the ligament fiber.

For the above bionic elastic fixing device, the width of the said elastic component is the same as that of the ligament fiber at the joint required to be fixed.

For the above bionic elastic fixing device, the said fixing assembly is mini-steel board. The mini-steel board is pressed on two ends of elastic band and fixed on the bone of dislocated joint by screws.

For the above bionic elastic fixing device, the said fixing assembly is a steel plate hook, which is connected to the elastic band and hooked at the edge of bone of dislocated joint.

For the above bionic elastic fixing device, the said fixing assembly is a screw, which fixes the elastic band on the bone of dislocated joint.

The present invention can achieve the following beneficial effects:

In the present invention, the elastic band is placed at the dislocated joint to be fixed, and fixed with fixing assembly on both ends. The elastic bands simulate the ligament tissue at the dislocated joint to fix the joint elastically. As compared with the traditional rigid fixation, it has the advantage of slight movement of the dislocated joint after fixing according to the biological characteristics of the original joint, maximally simulating the physiological state of the dislocated joints to achieve the optimal treatment effect; besides, it has such advantages as quick post-surgery recovery, low recurrence rate of dislocation and desirable functional recovery of the body part of the patient where dislocation occurs.

### BRIEF DESCRIPTION OF THE DRAWINGS

FIG. 1 is a schematic diagram of the bionic elastic fixing device;

FIG. 2 is another schematic diagram of the bionic elastic fixing device;

FIG. 3 is another schematic diagram of the bionic elastic fixing device;

FIG. 4 is a schematic diagram of the acromioclavicular joint in use;

FIG. 5 is a schematic diagram of the tibiofibula in use;

FIG. 6 is a schematic diagram of the symphysis pubis in use; and

FIG. 7 is a schematic diagram of the radius and ulna in use.

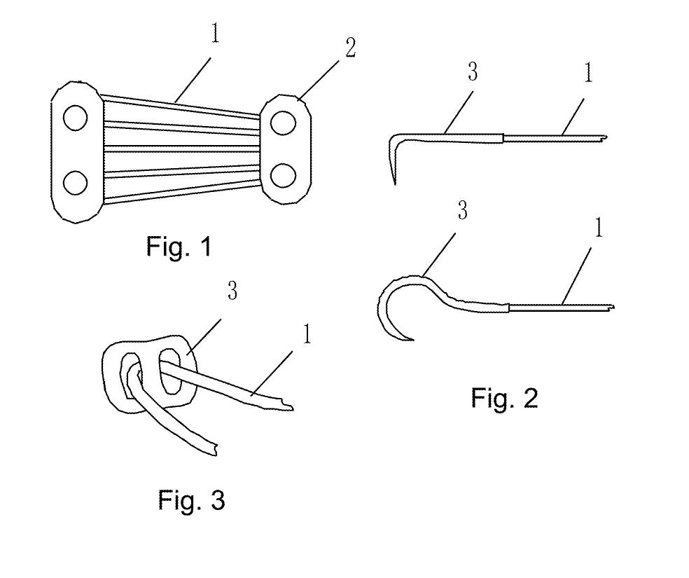

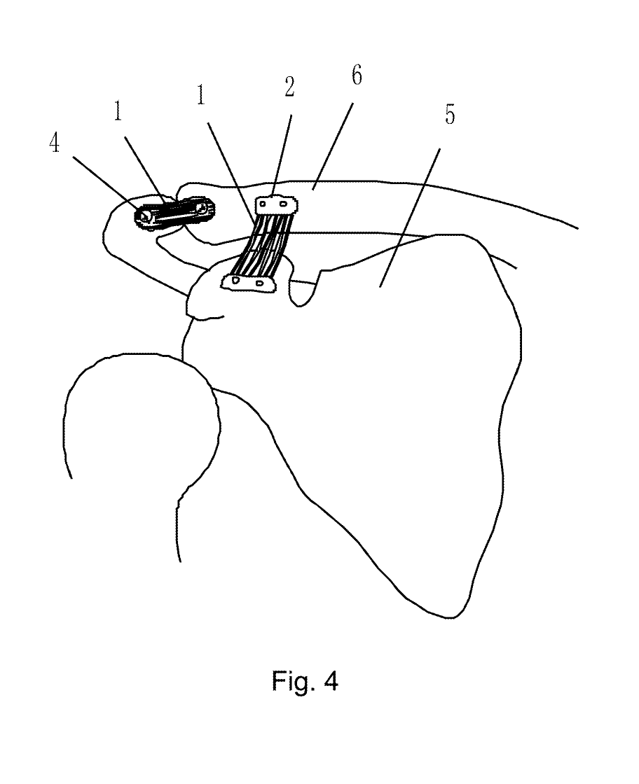

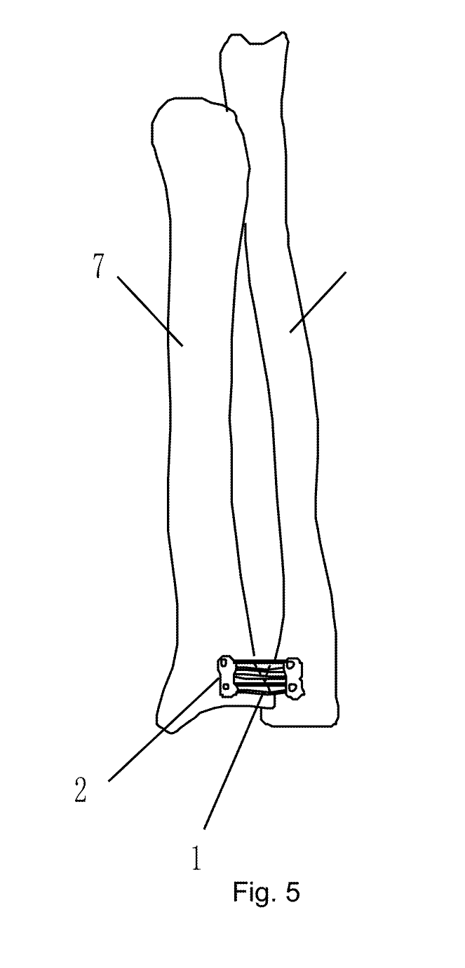

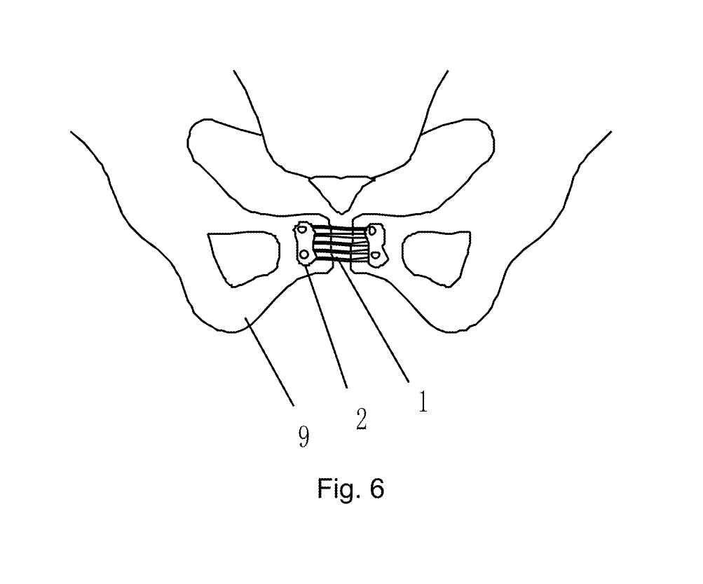

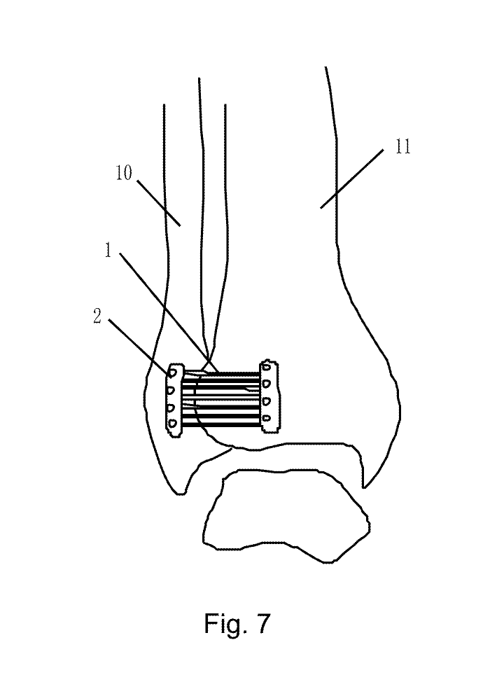

### DETAILED DESCRIPTION OF THE PREFERRED EMBODIMENTS

The invention is described herein in connection with drawings and certain specific embodiments. However, to the extent that the following detailed description is specific to a particular embodiment or a particular use, such is intended to be illustrative only and is not to be construed as limiting the scope of the invention. Through description, the advantages of the invention will become more explicit and easily understood.

The bionic elastic fixing device in the present invention consists of elastic component 1 and fixing assembly. The fixing assembly can be mini-steel board 2, or steel plate hook 3 or screw 4, which is to fix the elastic component 1 and dislocated joint.

As shown in the figure, elastic component 1 is strip-shaped, which can be multiple elastic cables, elastic mesh pieces or elastic bands; and their assembly direction is the same as the ligament fibers at the joint required to be fixed. The elastic modulus of the elastic component 1 matches the elastic modulus of the ligament fibers at the joint required to be fixed. In order to achieve a better bionic fixation effect, the starting and ending fixation points of the said elastic component 1 on the bone of dislocated joint are the same as the starting and ending growing points of the ligaments at the dislocated joint. The width of the said elastic component 1 is the consistent with that of the ligament fiber at the joint required to be fixed.

The elastic component 1 can be made from biopolymer materials, natural materials, such as collagen, cellulose, natural coral etc., or autologous tendon or allogeneic tendon, or various kinds of metal materials.

The two ends of the elastic component 1 are fixed on both ends of the dislocated joint by the fixing assembly.

As shown in the figure, the said fixing assembly is mini-steel board 2, which is pressed on two ends of elastic component 1 and fixed on the bone of dislocated joint by screws.

As shown in the figure, the said fixing assembly is steel plate hook 3, which is connected to the elastic component 1 and hooked at the edge of bone of dislocated joint.

As shown in the figure, the said fixing assembly is screw 4, which fixes the elastic component 1 on the bone of dislocated joint.

The bionic elastic fixing device in the present invention can be used for the internal fixation treatment of a variety of joint dislocations such as acromioclavicular joint dislocation, tibiofibular separation, symphysis pubis separation, radius and ulna separation, etc. The specific embodiments are described in details below.

The implementation in the treatment of acromioclavicular joint dislocation is described as follows.

The elastic component 1 is designed according to the fiber direction of coracoacromial ligament and coracoclavicular ligament and elastic modulus, which consists of 2-4 cables or sheet structures. The elastic component 1 can be fixed on scapula 5 and clavicle 6 on both ends of acromioclavicular joint by the mini-steel board 2 respectively, or can be hooked on the edges of bones of scapula 5 and clavicle 6 by the steel plate hook 3, or penetrated into distal clavicle through acromion and distal end of clavicle 6 through coracoid; after completing fixation, it can simulate the physiological state of the acromioclavicular joint, to achieve fixation of slight movement.

The implementation in the treatment of tibiofibular separation is described as follows.

The elastic component 1 is designed according to the fiber direction of anterior and posterior lower tibiofibular ligaments and the elastic modulus, which consists of 2-4 cables or sheet structures. The elastic component 1 can be fixed on both ends of tibia7, fibula 8 by the mini-steel board 2 respectively, or can be hooked on the edges of bones of tibia7, fibula 8 by the steel plate hook 3, or penetrated out from fibula 8 to tibia7. After completing fixation, it can simulate the physiological state of the inferior tibiofibular joint, to achieve fixation of slight movement.

The implementation in the treatment of symphysis pubis separation is described as follows.

The superior part of symphysis pubis is superior pubic ligament and the anterior part is the anterior pubic ligament. The elastic component 1 is designed according to the starting and ending points, direction and elastic modulus of the aforesaid ligaments, which consists of 2-4 cables or sheet structures. The elastic component 1 can be fixed on both ends of the left and right sides of pubis 9 by the mini-steel board 2 respectively, or can be hooked on the edges of bones of both sides of pubis 9 by the steel plate hook 3, or penetrated out from both sides of pubis 9 by screws and nuts. After completing fixation, it can simulate the physiological state of the symphysis pubis to achieve fixation of slight movement. Another elastic component 1 can also be designed according to the anterior pubic ligament.

The implementation in the treatment of distal radioulnar joint separation is described as follows.

The bionic elastic fixing device is designed according to the directions and shapes of distal radioulnar joint interosseous membrane, dorsal radioulnar ligament and palmar radioulnar ligament. One of the devices is the elastic component 1, which consists of 2-4 cables or sheet structure or mesh structures. The elastic component 1 can be fixed on both ends of the ulna10 and radius 11 by the mini-steel board 2 respectively, or can be hooked on the edges of bones of ulna10 and radius 11 by the steel plate hook 3 or screw hook, or penetrated out from the ulna10 to radius 11. After completing fixation, it can simulate the physiological state of the distal tibiofibular syndesmosis, to achieve fixation of slight movement. Another elastic component 1 is to simulate the palmar radioulnar ligament, and a third elastic component 1 is to simulate the interosseous membrane. The three elastic bands can be combined according to the injured parts and severity.

In summary, the key part the present invention is to set the elastic component according to the same direction and matching elasticity modulus with the ligament tissue at the dislocated joint to be fixed to fix the dislocated joint, to achieve slight movement of the dislocated joint after fixing according to the biological characteristics of the original joint, maximally simulate its physiological state, and get the optimal treatment effect. Therefore, the embodiment above cannot limit the protection scope of this invention on this account. Equivalent conversion or modification according to the spiritual essence of main technical scheme of this invention should be covered in the protection scope of the invention.

1. A bionic elastic fixing device, comprising an elastic component (1) and a fixing assembly that fixedly connects the two ends of the elastic component (1) and the dislocated joint required to be fixed; wherein the said elastic component (1) is of strip-shaped, and its assembly direction is the same as the ligament fibers at the joint required to be fixed, an elastic modulus of the elastic component (1) matches the elastic modulus of the ligament fibers at the joint required to be fixed, the fixing assembly is fixedly mounted to the two ends of the elastic component (1).

2. The bionic elastic fixing device according to claim 1, wherein the said elastic component (1) is multiple elastic cables, elastic mesh pieces or elastic bands.

3. The bionic elastic fixing device according to claim 2, wherein the length of the said elastic component (1) is the same as the length of ligament fiber at the joint required to be fixed, and the fixed points on two ends of elastic component (1) are the same as the growing points of the two ends of the ligament fiber; the width of the said elastic component (1) is the same as that of the ligament fiber at the joint required to be fixed.

4. The bionic elastic fixing device according to claim 1, wherein the said fixing assembly is mini-steel board (2); the mini-steel board (2) is pressed on two ends of elastic band (1) and fixed on the bone of dislocated joint by screws.

5. The bionic elastic fixing device according to claim 1, wherein the said fixing assembly is a steel plate hook (3), which is connected to the elastic band (1) and hooked at the edge of bone of dislocated joint.

6. The bionic elastic fixing device according to claim 1, wherein the said fixing assembly is a steel plate hook (3), which is connected to the elastic band (1) and hooked at the edge of bone of dislocated joint.

7. The bionic elastic fixing device according to claim 1, wherein the said fixing assembly is a screw (4), which fixes the elastic band (1) on the bone of dislocated joint.

8. The bionic elastic fixing device according to claim 2, wherein the said fixing assembly is mini-steel board (2); the mini-steel board (2) is pressed on two ends of elastic band (1) and fixed on the bone of dislocated joint by screws.

9. The bionic elastic fixing device according to claim 3, wherein the said fixing assembly is mini-steel board (2); the mini-steel board (2) is pressed on two ends of elastic band (1) and fixed on the bone of dislocated joint by screws.

10. The bionic elastic fixing device according to claim 2, wherein the said fixing assembly is a steel plate hook (3), which is connected to the elastic band (1) and hooked at the edge of bone of dislocated joint.

11. The bionic elastic fixing device according to claim 3, wherein the said fixing assembly is a steel plate hook (3), which is connected to the elastic band (1) and hooked at the edge of bone of dislocated joint.

12. The bionic elastic fixing device according to claim 2, wherein the said fixing assembly is a steel plate hook (3), which is connected to the elastic band (1) and hooked at the edge of bone of dislocated joint.

13. The bionic elastic fixing device according to claim 3, wherein the said fixing assembly is a steel plate hook (3), which is connected to the elastic band (1) and hooked at the edge of bone of dislocated joint.

14. The bionic elastic fixing device according to claim 2, wherein the said fixing assembly is a screw (4), which fixes the elastic band (1) on the bone of dislocated joint.

15. The bionic elastic fixing device according to claim 3, wherein the said fixing assembly is a screw (4), which fixes the elastic band (1) on the bone of dislocated joint.
